(19) United States
(12) Patent Application Publication
(10) Pub. No.: US 2014/0227212 A1
(43) Pub. Date: Aug. 14, 2014

(54) POLYMERIZED SUNSCREEN ABSORBERS

(71) Applicants: Kenneth B. Wagener, Gainesville, FL (US); Brian S. Aitken, Satellite Beach, FL (US); Kenneth B. Sloan, Gainesville, FL (US); Jason D. Heffley, Coral Springs, FL (US)

(72) Inventors: Kenneth B. Wagener, Gainesville, FL (US); Brian S. Aitken, Satellite Beach, FL (US); Kenneth B. Sloan, Gainesville, FL (US); Jason D. Heffley, Coral Springs, FL (US)

(73) Assignee: UNIVERSITY OF FLORIDA RESEARCH FOUNDATION, INC., GAINESVILLE, FL (US)

(21) Appl. No.: 14/343,985

(22) PCT Filed: Sep. 10, 2012
(86) PCT No.: PCT/US12/54376

§ 371 Date: Mar. 10, 2014

Related U.S. Application Data
(60) Provisional application No. 61/532,701, filed on Sep. 9, 2011.

(51) Int. Cl. A61K 8/81; A61Q 17/04

(52) U.S. Cl. CPC A61K 8/8164; A61Q 17/04
USPC 424/60; 424/59; 526/282; 558/402; 560/120

(57) ABSTRACT

A soluble UVA and/or UVB absorbing chromophore-comprising polymer useful for preparation of a sunscreen lotion has a multiplicity of repeating units where one or more of the repeating units includes at least one UVA and/or UVB absorbing chromophore. The repeating units are those formed from the ring-opening metathesis polymerization (ROMP) of a strained cycloakene, cycloalkadiene, bicycloalkene, or bicycloalkadiene, or where one or more sp³ hybridized carbons of the strained cycloakene, cycloalkadiene, bicycloalkene, or bicycloalkadiene is replaced with a heteroatom. The UVA and/or UVB absorbing chromophore-comprising polymer can be formed from a UVA and/or UVB absorbing chromophore-comprising monomer, which can be homopolymerized or copolymerized by ROMP. Alternately, the UVA and/or UVB absorbing chromophore-comprising polymer can be formed by polymerization of monomers that can subsequently be substituted with UVA and/or UVB absorbing chromophores.

### CROSS-REFERENCE TO RELATED APPLICATION

The present application claims the benefit of U.S. Provisional Application Ser. No. 61/532,701, filed Sep. 9, 2011, which is hereby incorporated by reference herein in its entirety, including any figures, tables, or drawings.

The subject invention was made with government support under Contract No. DMR-0703261, awarded by the National Science Foundation. The government has certain rights in the invention.

### BACKGROUND OF INVENTION

UV radiation penetrates the ozone layer over two wavelength regimes, UVB (290-320 nm) and UVA (320-400 nm). UVB acts directly on biological molecules, causing the familiar delayed sunburn that arises 12-24 hours after exposure, skin aging, skin cancer (melanoma), and eye photokeratities. UVA acts indirectly with the skin by forming reactive oxygen species, causing “immediate” sunburn that diminishes within 2 hours after exposure. UVA potentially plays a role in delayed sunburn and skin cancer. Although less energetic,

UVA, which accounts for about 5.6% of sunlight, penetrates the skin more deeply, even into the dermis, than does UVB radiation, which is about 0.5% of sunlight and, generally, is limited to the epidermis.

Melanoma has experienced the most rapid increase in the number of cases of all forms of cancer, with more than 51,000 cases reported in America each year. As most cases have been attributed to sun exposure, the market for sunscreens presently exceeds $1 billion a year. Sunscreens are substances used to protect the skin by absorbing, reflecting and/or scattering damaging ultraviolet (UV) radiation. Sunscreens are typically used as a component in a cream or lotion. Sunscreen formulations are an article intended for the prevention of a disease and are regulated as an over-the counter (OTC) drug.

In a Final Monograph of May 21, 1999, entitled “Sunscreen Drug Products for Over-The-Counter Human Use” by the U.S. Food and Drug Administration (FDA), conditions were established under which OTC sunscreen drug products are generally recognized as safe and effective and not misbranded as part of FDA's ongoing review of OTC drug products. The monograph established that an active ingredient of sunscreen products consists of any of the following (within the concentration specified for each ingredient when the finished product provides a minimum SPF value of not less than 2 as measured by a testing procedures established in the monograph): Aminobenzoic acid (PABA) (15%); Avobenzone (3%); Cinoxate (3%); Dioxybenzone (3%); Homosalate (15%); Menthyl anthranilate (5%); Octocrylene (10%); Octyl methoxycinnamate (7.5%); Octyl salicylate (5%); Oxybenzone (6%); Padimate O (8%); Phenylbenzimidazole sulfonic acid (4%); Sulisobenzone (10%); Titanium dioxide (25%); Trolamine salicylate (12%); and Zinc oxide (25%). Since 1999

Ecamsule (10%) and Phenylbenzimidazole sulfonic acid (4%) have been approved for use as sunscreens. Also approved for use in Europe are: 4-Methylbenzylidene camphor (4%); Bisoctrizole (10%); Bemotrizinol (10%); Bisdisulizole disodium (10%); Drometrizole trisiloxane (15%); Benzophenone-9 (10%); Ethylhexyl triazone (5%); Diethylamino hydroxybenzoyl hexyl benzoate (10%); Iscotrizinol (10%); Polysilicone-15 (10%); and Isoamyl p-Methoxycinnamate (10%).

Of these sunscreens, only Polysilicone-15 is a silicon based polymeric sunscreen with the IUPAC name α-(trimethylsilyl)-ω-(trimethylsilyloxy)poly[oxy(dimethyl)silylene]-co-[oxy-(methyl)(2-{4-[2,2-bis(ethoxycarbonyl)vinyl]phenoxy}-1-methyleneethyl)silylene]-co-[oxy-(methyl)(2-(4-[2,2-bis(ethoxycarbonyl)vinyl]phenoxy)prop-1-enyl)silylene] with about oxy(dimethyl)silylene units, about 4 oxy(methyl)(2-{4-[2,2-bis(ethoxycarbonyl) vinyl]phenoxy}-1-methyleneethyl) silylene units and about 1 oxy(methyl)(2-(4-[2,2-bis(ethoxycarbonyl) vinyl]phenoxy)prop-1-enyesilylene units on average with an average molecular weight of 6,070. Statistically, the random copolymer should have more than one percent of the chains that have no UV absorbing chromophores. An equivalent polymer of about 1,000 molecular weight would have less than half of the chains containing any UV absorbing chromophores. Polymeric sunscreens have the potential to provide a simple mode of distribution of the sunscreen in a vehicle, and to reduce or eliminate absorption of the sunscreen by the skin to which it is applied. Many examples of polymeric sunscreens have been disclosed in the patent literature.

U.S. Pat. Nos. 7,291,322, 6,376,679, 6,312,673, 6,251,373, 6,221,343, 6,214,324, 6,200,557, 6,159,456, and 5,753,209, and U.S. Patent Application Publications 2007/0020204, 2004/0213746, 2002/0054860, and 2001/0026789 are directed to silicone based polymeric sunscreens that are random copolymers similar to Polysilicone-15 with various UV absorbing chromophores. Another form of polymeric sunscreens is random acrylic copolymers, as disclosed in U.S. Pat. Nos. 5,741,924, 5,487,885, 5,099,027 and 4,524,061 (which also discloses the polymers from a cyclic lactams). Substantive polymers that are prepared by random vinyl copolymerization are disclosed in U.S. Pat. No. 7,087,692 and U.S. Patent Application Publication 2004/0101498. Substantive polymers that are prepared by random condensation copolymerization are disclosed in U.S. Pat. Nos. 4,004,074 and 3,864,473. Water dispersible polymeric sunscreens have been prepared by a random condensation copolymerization with polyethylene glycol monomers, as disclosed in U.S. Pat. Nos. 5,250,652, 5,243,021, and 5,134,223. An acrylamide homopolymer having UV active chromophores at every repeating unit is disclosed in U.S. Pat. No. 4,233,430. U.S. Patent Application Publication 2005/0186152 discloses a polyanhydride modified by the addition of nucleophilic UV active chromophores to place the chromophores on every repeating unit of the polymer chain with the formation of an equal amount of carboxylic acid groups on the polymer chain. U.S. Pat. Nos. 6,962,692, 6,926,887, 6,919,473, 6,899,866, 6,890,521, 6,800,274, and 5,993,789, disclose a homo-polyester sunscreen where UV active chromophores are on every repeating unit of the polymer.

As disclosed, polymeric sunscreens have very high levels of UV absorbing chromophores that can result in a non-uniform distribution of the chromophores in the vehicles, as can occur with homopolymer where every repeating unit has the chromophore, or with random copolymers where molecular weights or UV absorbing chromophores are limited to those having high molecular weights or high levels of UV absorbing units. A method of making uniform molecular weight homopolymers or copolymers having similar quantities of chromophores on every chain would be advantageous for the preparation of a sunscreen product that has enhanced chromophore stability and that is resistant to skin penetration.

### BRIEF SUMMARY

Embodiments of the invention are directed to soluble polymeric sunscreens where the polymer has a narrow molecular weight distribution and has one or more repeating units that comprise a chromophore unit that is an effective sunscreen for UVA and/or UVB. The UV absorbing chromophores can be equivalents to and derived from the conjugated group of the approved sunscreens: Aminobenzoic acid; Avobenzone; Cinoxate; Dioxybenzone; Homosalate; Menthyl anthranilate; Octocrylene; Octyl methoxycinnamate; Octyl salicylate; Oxybenzone; Padimate O; Phenylbenzimidazole sulfonic acid; Sulisobenzone; Trolamine salicylate; Ecamsule; Phenylbenzimidazole sulfonic acid; 4-Methylbenzylidene camphor; Bisoctrizole; Bemotrizinol; Bisdisulizole disodium; Drometrizole trisiloxane; Benzophenone-9; Ethylhexyl triazone; Diethylamino hydroxybenzoyl hexyl benzoate; Iscotrizinol; or Isoamyl p-Methoxycinnamate. The polymer is substituted with the chromophores, which can occur because the chromophores are attached to a monomer before polymerization to the polymeric sunscreen, or it can be formed on a prepolymer that has a reactive functionality on at least one repeating unit, where the chromophore is attached by reaction of a complementary functionality with the reactive functionality on the repeating unit(s) of the prepolymer. Once prepared the UVA and/or UVB absorbing chromophore-comprising polymer can be transformed to a second UVA and/or UVB absorbing chromophore-comprising polymer by reaction with the polymer, for example, reaction with the alkene groups in the chain that result from a ring-opening metathesis polymerization (ROMP)

Embodiments of the invention are directed to monomers for the preparation of the above UVA and/or UVB absorbing chromophore-comprising polymer. The monomers comprise a cycloalkane, which polymerizes by a (ROMP) mechanism and one or more UV absorbing chromophores that absorb light in the UVA and/or UVB regions of the electromagnetic spectrum. In an embodiment of the invention, the cycloalkane ring undergoes an exothermic ring-opening during ROMP and the polymerization is “living” in nature, where the polymerization occurs to a large extent with little or no chain-transfer to a polymer chain to yield a narrow molecular weight distribution and high, often quantitative, conversion of the monomer. In embodiments of the invention, the UV absorbing chromophore or chromophores are attached to the monomer ring such that polymerization yields a polymer that is not high symmetric and is amorphous, such that it has a high solubility in at least one solvent that is useful in a personal care formulation. In another embodiment of the invention, a copolymer is produced between two or more cycloalkane- comprising monomers such that an amorphous copolymer is formed. The copolymer can comprise one or more monomers comprising one or more UV absorbing chromophores and can comprise other cycloalkane-comprising monomers that lack a chromophore, but may include other units to promote solubility or other properties, such as those desired for a pleasant feel and appearance on a skin surface.

Other embodiments of the invention are directed to a method of preparing a polymeric sunscreen, where one or more of the cycloalkane-comprising monomers are polymerized in the presence of a catalyst to promote ROMP and where the degree of polymerization can be controlled. Olefin metathesis catalysts that can be used include Schrock's catalyst or Grub's catalyst.

Other embodiments of the invention are directed to sunscreen lotions where the polymeric sunscreens are included with a fluid vehicle. The fluid vehicle can include a solvent for the polymeric sunscreen or can include any combination of solvents, non-solvents, and/or dispersing agents to emulsify or suspend the polymeric sunscreen in a non-solvent vehicle.

Another embodiment of the invention is a method to prevent sunburn by providing a polymeric sunscreen that is applied to skin. The polymeric sunscreen can be delivered to the skin as a component of a fluid that is a solution, emulsion, and/or dispersion.

### BRIEF DESCRIPTION OF DRAWINGS

FIG. 1 shows a reaction scheme for the preparation of a UVA and/or UVB absorbing chromophore-comprising monomer, according to an embodiment of the invention, where the chromophore is derived from Avobenzone.

FIG. 2 shows a reaction scheme for the preparation of a UVA and/or UVB absorbing chromophore-comprising monomer, according to an embodiment of the invention, where the chromophore is derived from Octocrylene.

FIG. 3 shows a reaction scheme for the homopolymerization of the monomers illustrated in FIG. 1, bottom, and FIG. 2, top, by ring-opening metathesis polymerization, according to embodiments of the invention, to form UVA and/or UVB absorbing chromophore-comprising polymers, specifically an Avobenzone equivalent comprising polymer, top, and an Octocrylene equivalent comprising polymer, bottom, according to embodiments of the invention.

FIG. 4 shows a reaction scheme for the copolymerization of the monomers illustrated in FIG. 1 and FIG. 2 by ring-opening metathesis polymerization, according to an embodiment of the invention, to form a UVA and/or UVB absorbing chromophore-comprising polymer, a copolymer, according to an embodiment of the invention.

FIG. 5 shows a reaction scheme for the copolymerization of three monomers by ring-opening metathesis polymerization, according to an embodiment of the invention, to form a UVA and/or UVB absorbing chromophore-comprising polymer, a terpolymer, according to an embodiment of the invention.

FIG. 6 shows a reaction scheme for the copolymerization of two monomers by ring-opening metathesis polymerization, according to an embodiment of the invention, to form a protected prepolymer for subsequent transformations by deprotection and simultaneous substitutions, according to an embodiment of the invention, to form a UVA and/or UVB absorbing chromophore-comprising polymer, a terpolymer, according to an embodiment of the invention.

FIG. 7 shows a reaction scheme for the transformation of the UVA and/or UVB absorbing chromophore-comprising polymer, a terpolymer, according to an embodiment of the invention, formed as shown in either FIG. 5 or FIG. 6, to a hydrogenated UVA and/or UVB absorbing chromophore-comprising polymer, a terpolymer, according to an embodiment of the invention.

FIG. 8 shows the preparation of a UVB absorbing chromophore-comprising polymer, according to an embodiment of the invention, by polymerization of norbornene by ROMP followed by the addition of water and esterification with a chromophore derived from Octocrylene to form a UVB absorbing chromophore-comprising polymer according to an embodiment of the invention.

### DETAILED DISCLOSURE

Embodiments of the invention are directed to polymeric sunscreens that comprise a polymeric backbone that has a narrow distribution of repeating units where at least one of the repeating units of the chain comprises one or more UVA and/or UVB absorbing chromophores. Sunscreen molecules that comprise a single UVA and/or UVB absorbing chromophores of relatively low molecular weight are often absorbable by the skin. Skin absorption can allow radical/oxidative damage to the skin or can be toxic or carcinogenic to the sunscreen user. A photolytically and hydrolytically stable polymeric structure prohibits the skin absorption of the individual UV absorbing chromophores that function as the sunscreen. The polymeric sunscreen is soluble in at least one solvent that is useful as a fluid vehicle in a personal care formulation. In embodiments of the invention, the polymeric sunscreen comprises a polymer or copolymer of low symmetry such that crystallization is inhibited and solubility is achievable in a desirable solvent to provide a sunscreen lotion useful for application to skin for protection against sunburn.

In one embodiment of the invention, the polymeric sunscreen is a polymer that can be formed by a ring-opening metathesis polymerization (ROMP) of a cycloalkane-comprising monomer, where the resulting polymer has at least one repeating unit to which one or more UVA and/or UVB absorbing chromophores are attached. The chromophore can be attached to a monomer before polymerization, or can be attached by a reaction with the polymer after polymerization. In an embodiment of the invention, the cycloalkane-comprising monomer undergoes an exothermic ring-opening, such that chain-transfer to polymer or chain-chain redistribution is low, often absent, relative to the ring-opening polymerization process, which promotes a narrow molecular weight distribution and very high, often quantitative, conversion of the monomers. In an embodiment of the invention, because the polymerization process results in a Poisson distribution of repeating units rather than a normal or broader distribution, a relatively low molecular weight polymeric sunscreen, an oligomeric sunscreen, can be formed that lacks a significant fraction of the distribution that is monomeric, often free of a monomeric species. According to an embodiment of the invention, the ratio of the weight average to number average degree of polymerization, the molecular weight distribution, can be less than 1.5, for example, 1.4, 1.35, 1.3, 1.25, 1.2, 1.15, 1.1, 1.05 or 1.01. Such an oligomeric sunscreen can display a heightened miscibility with a fluid vehicle for a sunscreen lotion and permit a relatively high concentration of UVA and/or UVB absorbing chromophores in a lotion that would otherwise display an undesirably high viscosity because polymeric sunscreens from other routes require a higher degree of polymerization to assure almost no monomeric components in the sunscreen, which may be absorbable by the skin. The average degree of polymerization can be three or more, for example, about 3, 3.5, 4, 5, 6, 10, 20, 30, 40, 50, 100, 500, 1,000, or more.

In one embodiment of the invention, the polymer comprises a multiplicity of repeating units that have alkene units that reside at both ends of the repeating unit, and where at least one repeating unit comprises one or more UVA and/or UVB absorbing chromophores. In one embodiment of the invention, the polymeric sunscreen consists of a homopolymer from a single chromophore-comprising repeating unit or the polymeric sunscreen consists of a copolymer having two or more different chromophore-comprising repeating units, but where all repeating units comprise at least one UVA and/or UVB absorbing chromophore. In another embodiment of the invention, the polymeric sunscreen is a copolymer consisting of a multiplicity of repeating units, where at least one repeating unit comprises one or more INA and/or UVB absorbing chromophores and at least one repeating unit that lacks any UVA and/or UVB absorbing chromophores. A portion of the repeating units lacking any UVA and/or UVB absorbing chromophores can be an unsubstituted repeating unit and a portion of the repeating units lacking any UVA and/or UVB absorbing chromophores can be substituted to impart a desired property to the sunscreen lotion formed from the polymeric sunscreen. For example, one or more of the substituted repeating units lacking any UVA and/or UVB absorbing chromophores can include substituents to impart solubility in a desired solvent or to impart desired properties to the sunscreen lotion, such as, a color or a tactile sensation, as might be identified by a sensory panel during the development of a sunscreen lotions formulation.

In an embodiment of the invention, the repeating unit can be one that is derived from a cycloalkene, non-conjugated cycloalkadiene, bicycloalkene, or bicycloalkadiene having a ring that comprises at least three carbon atoms, and wherein the alkene has a “ring strain” and will polymerize exothermically. For example, a cycloalkane or cycloalkadiene from which the repeating unit is derived can have the structure:

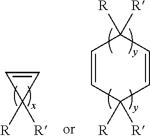

to yield repeating groups

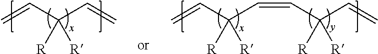

where x is 1 to 6, y is 1 or 2, and R and R′ are independently H, a substituent comprising a UVA and/or UVB absorbing chromophore, or any stable monovalent substituent, where any pair of R and R′ on the same carbon or different carbons of the cycloalkene or cycloalkadiene can be connected by a divalent substituent to form a second ring comprising three to twelve atoms forming a bicycloalkene or bicycloalkadiene, and where one or more of the sp³ hybridized carbons can be a replaced with a heteroatom, such as O, N, or Si. For example, the bicycloalkene with a heteroatom can be derived from 7-oxo-norbornene, norbornene-2,3-dicarboxylic anhydride, or any norbornene-2,3-dicarboxylic imide. In a specific embodiment of the invention, the repeating unit is derived from norbornene:

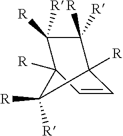

that upon ROMP results in the repeating unit:

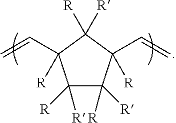

Where the polymeric sunscreen is a copolymer of two or more repeating units, the repeating units can be distributed randomly throughout the copolymer, or can be formed in a block or gradient manner where one or more of the repeating units dominate an initial end of the copolymer and one or more of the repeating units dominate the terminal end of the copolymer. For example the polymerization can be carried out with a continuous feed of the comonomers, where monomer addition to the active center is rapid relative to termination of the active center and the comonomer feed is controlled and changing during polymerization, or where the comonomers are of sufficiently different reactivity that one or more comonomers undergo addition to the active center at a significantly higher rate than do one or more other comonomers. For example, where addition is not diffusion controlled and the ring strain is significantly different for two or more comonomers, promoting the polymerization of one comonomer can be followed by polymerization of a second monomer or one or more additional comonomers in the initial comonomer mixture to form a block copolymer.

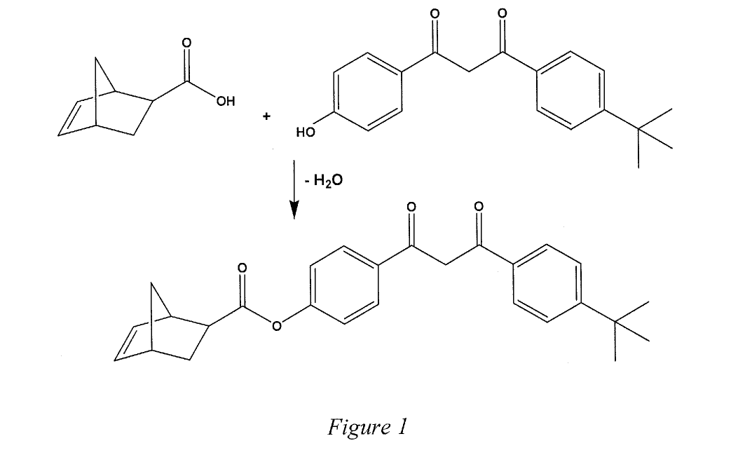

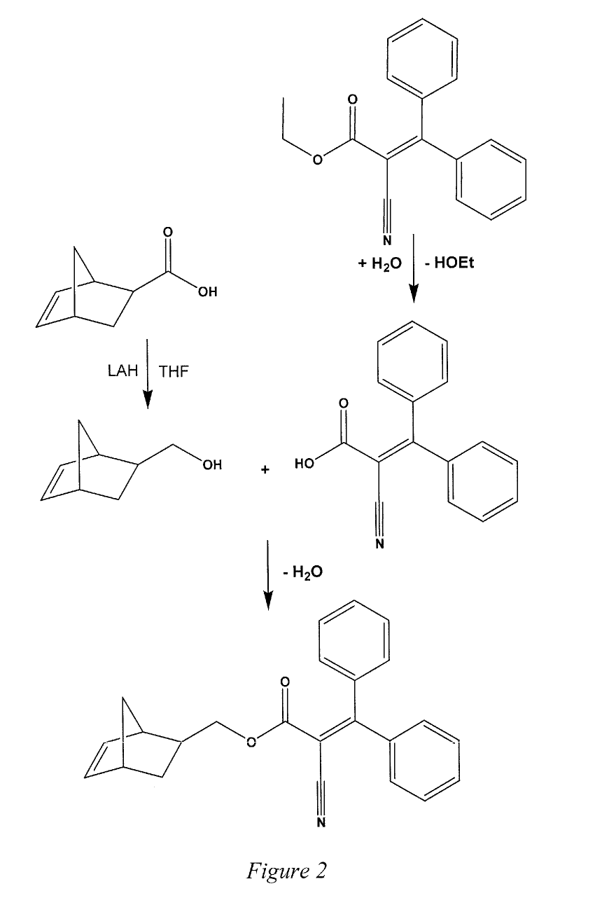

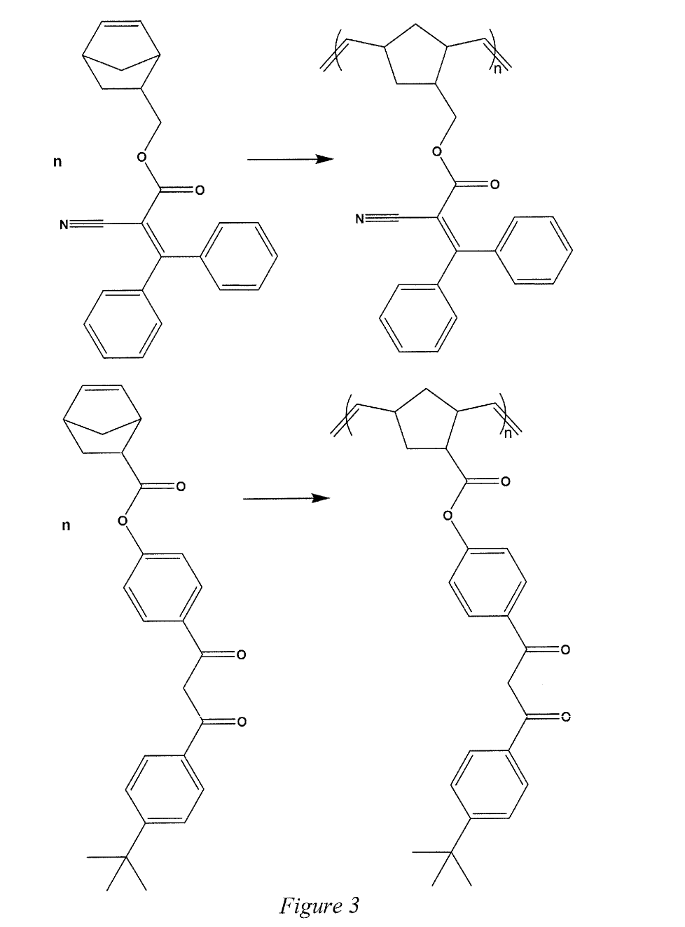

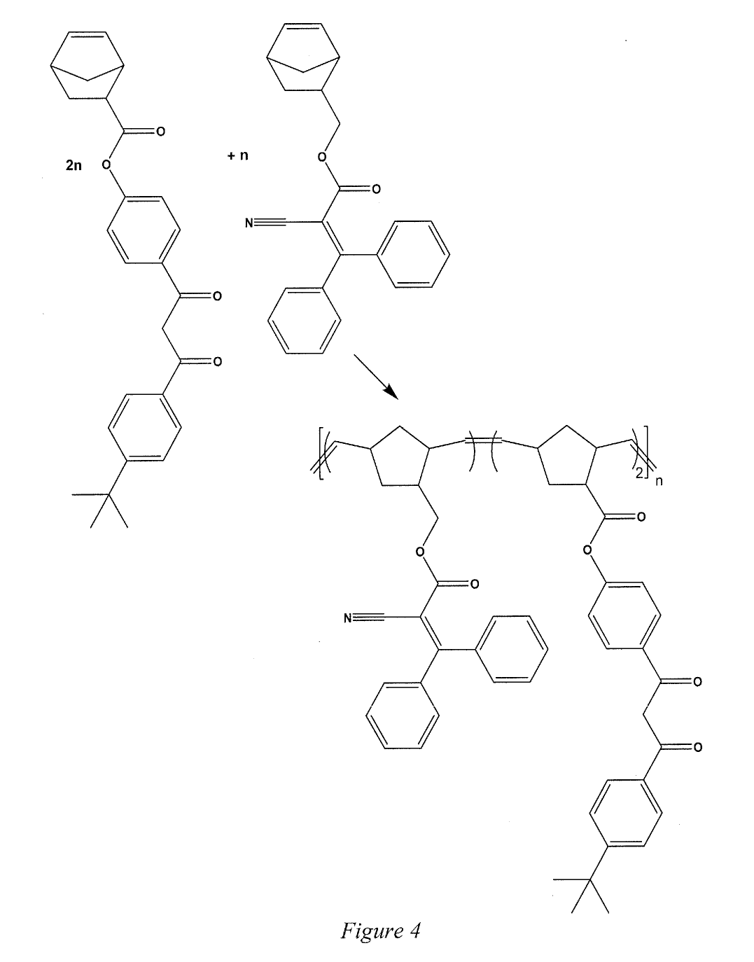

In exemplary embodiments of the invention, bicyclo[2.2.1]hept-5-ene-2-carboxylic acid can be converted into one or more UVA and/or UVB absorbing chromophore-comprising monomers, as illustrated by the reaction schemes in FIG. 1 from Avobenzone and in FIG. 2 for Octocrylene. Subsequently, these monomers can be homopolymerized, as illustrated in FIG. 3, or copolymerized, as illustrated for a 2:1 Avobenzone-comprising monomer: Octocrylene-comprising monomer mixture in FIG. 4, to form polymeric sunscreens according to embodiments of the invention. In an exemplary polymerization of the Avobenzone-comprising monomer, a polymer with a molecular weight of 9,100 g/mole (DP=22) and a polydispersity index of 1.3 resulted. In an exemplary polymerization of the Octocrylene-comprising monomer, a polymer with a molecular weight of 9,000 g/mole (DP=25) and a polydispersity index of 1.1 resulted. Also, a copolymerization of the 2:1 Avobenzone-comprising monomer:Octocrylene-comprising monomer mixture yielded a random copolymer with a molecular weight of 10,400 g/mole (DP=26) and a polydispersity index of 1.3. The polymers and copolymers from 2-substituted or 2,3 disubstituted norbornenes are not stereoregular polymers, but have a random stereochemistry where the chirality of the 2 or 3 positions can be different, the substituents' relative orientation, the tacticity, can vary, the geometric orientation, head-to-head, tail-to-tail, or head-to-tail, can vary, and both cis and trans geometries can result about the internal alkenes between repeating units. Hence, the resulting polymers and copolymers are not crystalline, which is advantageous for solubility in an acceptable solvent for formation of a sunscreen lotion.

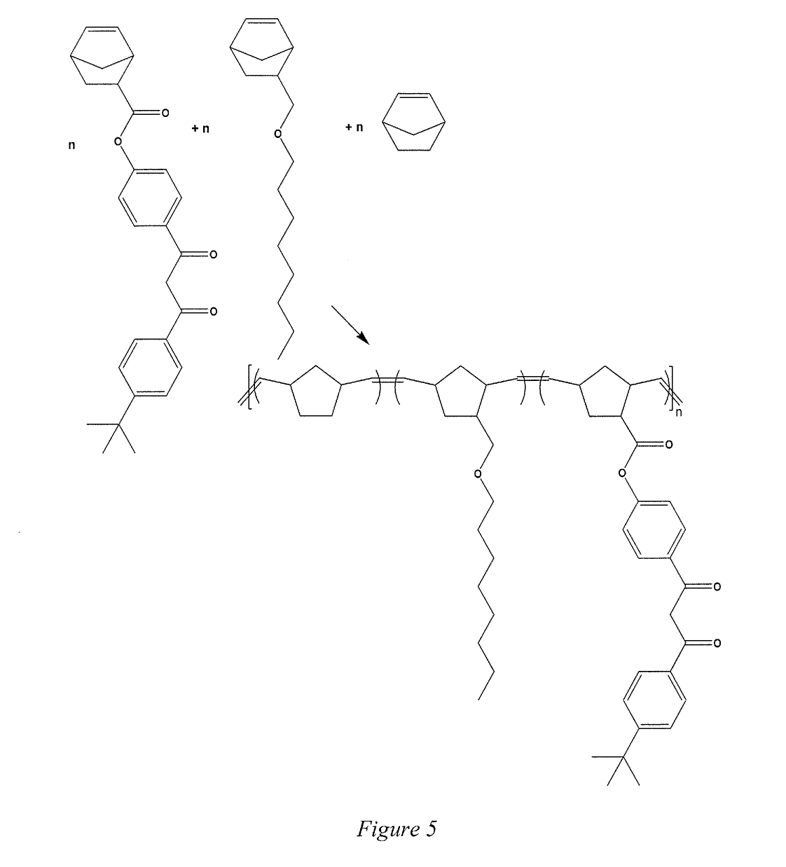

In other embodiments of the invention, the UVA and/or UVB absorbing chromophore-comprising monomers can be copolymerized with one or more monomers that do not provide a UVA and/or UVB absorbing chromophore, as illustrated in FIG. 5. The additional monomer or monomers are those that will readily copolymerize with the UVA and/or UVB absorbing chromophore-comprising monomers, but provide a desirable effect, for example, dilution of the chromophores in the polymer, enhancement of the solubility in a desired solvent, or attainment of a desired sensory effect for the sunscreen lotion formulated with the copolymer. The structures of substituents that can be included in copolymers are readily appreciated by those skilled in the art, and include, but are not exclusive to: alkyl chains, tetraalkyl ammonium salts, poly or oligo(ethyleneoxide) chains, or di- or trialkoxysilane functionality.

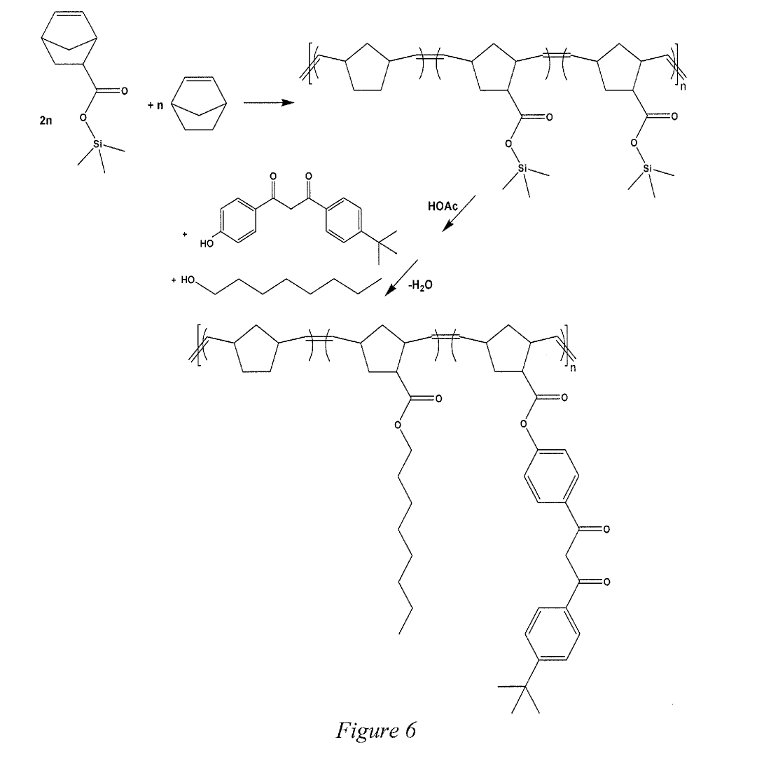

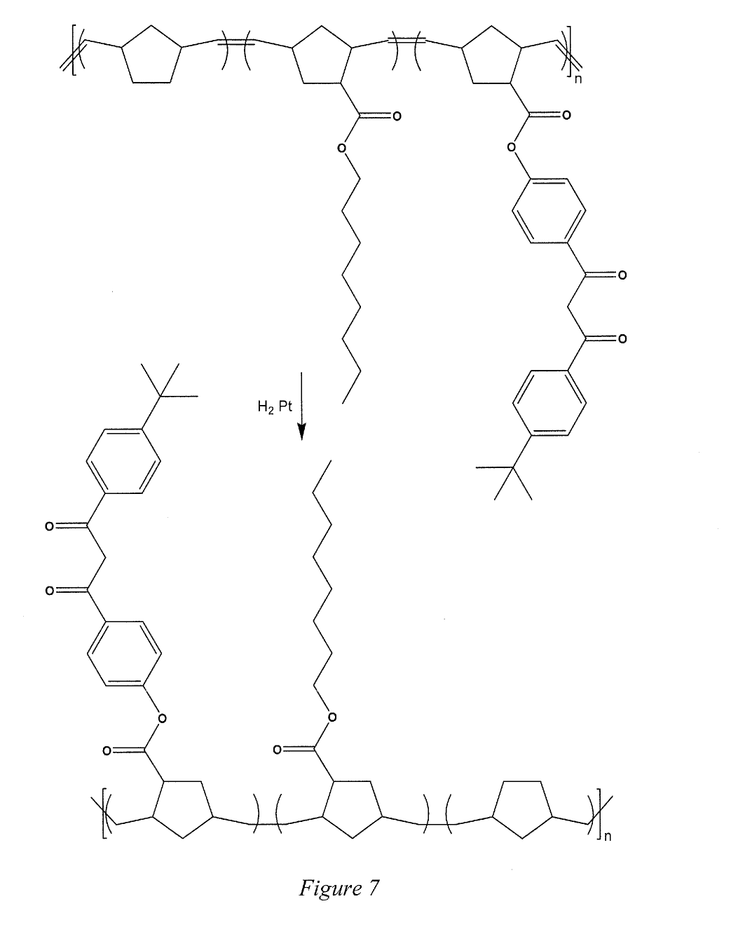

In addition to polymerization and copolymerization of UVA and/or UVB absorbing chromophore-comprising monomers, monomers free of UVA and/or UVB absorbing chromophore can be polymerized by ROMP to a polymer that can subsequently be converted into a soluble UVA and/or UVB absorbing chromophore-comprising polymer, according to embodiments of the invention. For example, a silyl ester of bicyclo[2.2.1]hept-5-ene-2-carboxylic acid can be polymerized or copolymerized, deprotected, and subsequently transformed into a UVA and/or UVB absorbing chromophore-comprising polymer, as indicated in FIG. 6, for the preparation of a terpolymer. In other embodiments of the invention, the polymer resulting from ROMP can be converted into a reduced or otherwise substituted polymer by reaction of the alkene groups between repeating units. As illustrated in FIG. 7, the UVA and/or UVB absorbing chromophore-comprising polymer can undergo reduction of the alkene. In other embodiments of the invention, the alkene can be converted into the site of substitution of a UVA and/or UVB absorbing chromophore, as illustrated in

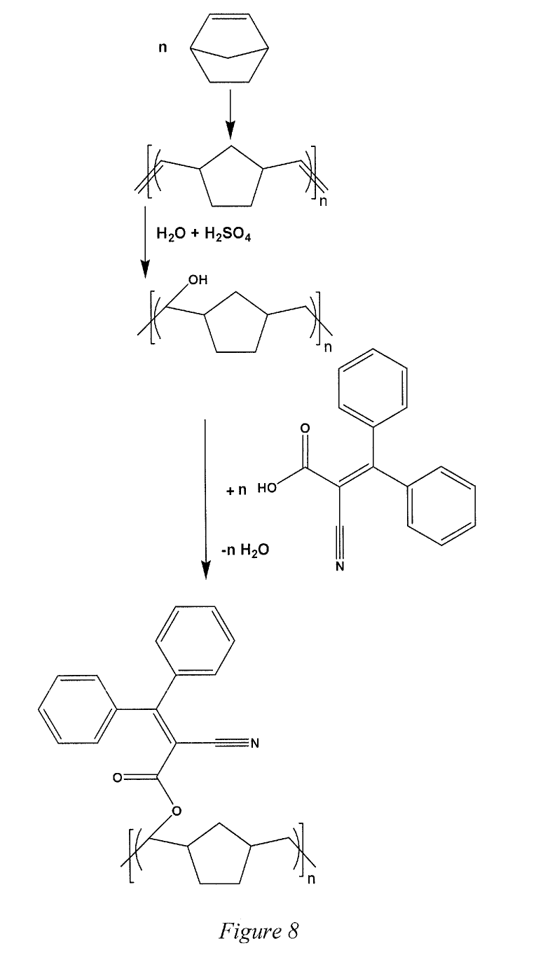

FIG. 8. Advantageously, the polymer reactions, unlike the full conversion limit shown in FIG. 8, need not be carried out to functionalize every repeating unit of the polymer to form a desirable UVA and/or UVB absorbing chromophore-comprising polymer.

The types of UVA and/or UVB absorbing chromophores can be, but are not limited to, those that have equivalent conjugated structures to the compounds approved for use by the

FDA or other worldwide regulatory agencies. Those approved chromophores are: Aminobenzoic acid; Avobenzone; Cinoxate; Dioxybenzone; Homosalate; Menthyl anthranilate; Octocrylene; Octyl methoxycinnamate; Octyl salicylate; Oxybenzone; Padimate O; Phenylbenzimidazole sulfonic acid; Sulisobenzone; Trolamine salicylate; Ecamsule; Phenylbenzimidazole sulfonic acid; 4-Methylbenzylidene camphor; Bisoctrizole; Bemotrizinol; Bisdisulizole disodium; Drometrizole trisiloxane; Benzophenone-9; Ethylhexyl triazone; Diethylamino hydroxybenzoyl hexyl benzoate; Iscotrizinol; Isoamyl p-Methoxycinnamate; and those attached to Polysilicone-15. Other UV absorbing chromophores can be used including those equivalent to UV absorbing conjugated systems disclosed in: U.S. Pat. Nos. 7,291,322; 7,087,692; 6,962,692; 6,926,887; 6,919,473; 6,899,866; 6,890,521; 6,800,274; 6,376,679; 6,312,673; 6,251,373; 6,221,343; 6,214,324; 6,200,557; 6,159,456; 5,993,789; 5,753,209; 5,741,924; 5,487,885; 5,250,652; 5,243,021; 5,134,223; 5,099,027; 4,524,061; 4,233,430; 4,004,074; and 3,864,473 and U.S. Patent Application Publications 2007/0020204; 2005/0186152; 2004/0213746; 2004/0101498; 2002/0054860; and 2001/0026789. Many other conjugated systems that absorb in the UVA and/or UVB spectral ranges can be used, even some that are known to be damaging to the skin or to other organs or systems of an individual using a sunscreen because the chromophores are irreversibly bound to a polymer chain that renders it passive to the system. The UV absorbing chromophores can be linked in the chromophore unit by any sufficiently stable bridging group. For example, where the conjugated UV absorbing chromophore unit contains a carboxylic acid group, the chromophore unit can have a hydroxy group and the UV absorbing chromophores can be attached via ester functionality. The bridging group can be a single bond or it can be a series of covalently bonded atoms, depending on what is required to link the chromophore to the cycloalkene or cycloalkadiene moiety. One skilled in the art can readily appreciate appropriate complimentary functionalities to form desirable bridges between the UV absorbing chromophores to polymerizable moieties of the monomers.

The formation of UVA and/or UVB absorbing chromophore-comprising polymer can be carried out by polymerization of the strained cycloakene, cycloalkadiene, bicycloalkene, or bicycloalkadiene comprising monomers using any known metathesis catalyst, for example Schrock's catalyst Mo(═CHCMe₂Ph)(N-2,6-C₆H₃-i-Pr₂)(OCMe(CF₃)₂)₂ or Grubbs' catalyst RuC1₂(═CHPh)(PCy₃)₂, where the specific catalyst that is employed depends upon the structure of the monomers used, as taught in Trnka et al. Acc. Chem. Res. 2001, 34, 18-29, and Handbook of Metathesis, Grubbs, R. H., Ed.; Wiley-VCH: Weinheim, Germany, 2003 and can be appreciated by those skilled in the art. As needed, the UVA and/or UVB absorbing chromophore-comprising polymer can be isolated from the polymerization mixture by any method, including precipitation, extraction, or centrifugation.

The catalyst that is used to prepare the polymer depends upon the UVA and/or UVB absorbing chromophores that are present in the monomer. For example, the presence of a chromophore equivalent in structure to Octocrylene in a monomer, as shown in FIG. 2, results in a polymer, as shown in FIG. 3, that does not easily permit the removal of a ruthenium based catalyst, such as Grubbs' catalyst. However, the monomer comprising an Octocrylene equivalent can be polymerized by Schrock's catalyst and easily purified. In contrast, polymerization of a monomer comprising an Avobenzone equivalent chromophore, as shown in FIG. 3, is readily polymerized by Grubbs' catalyst, but resists polymerization by Schrock's catalyst.

Once in possession of UVA and/or UVB absorbing chromophore-comprising polymer in a desired isolated form, the polymer can be combined with one or more solvents, and, as needed or desired, a surfactant, emulsifier, fragrance, buffers, powder, or any other ingredient to form a lotion or other fluid vehicle for application to skin in need of sun protection. The proportions of the UVA and/or UVB absorbing chromophore-comprising polymer and other ingredients are chosen to give the desired degree of sun protection, for example a desired SPF number, to achieve a desired flow, and to provide other properties that are conducive with a mode of manufacture or to the desired sensory properties of the fluid when spread and maintained on the skin.

### METHODS AND MATERIALS

### Synthesis of 2-cyano-3,3-diphenylacrylic Acid

As indicated in FIG. 2, 10 g ethyl 2-cyano-3,3-diphenylacrylate dissolved in 100 mL of 50:50 ethanol:water was placed in a 250 mL round bottom flask equipped with a reflux condenser. After addition of 10 g potassium hydroxide the mixture was refluxed overnight and 1 M aq. HCl was added dropwise until the pH was approximately 2. The solution was extracted twice with 50 mL portions of ethyl acetate and the organic layer was dried over anhydrous magnesium sulfate and evaporated to yield 8.8 g of 2-cyano-3,3-diphenylacrylic acid.

### Synthesis of Octocrylene Equivalent Monomer

As indicated in FIG. 2, under a nitrogen atmosphere, a round bottom flask was charged with 7.5 g of 2-cyano-3,3-diphenylacrylic acid (30.09 mmol), 5.6 g of 5-norbornene-2-methanol (mixture of isomers, predominantly endo, 45.13 mmol, 1.5 eq.), 9.3 g dicyclohexylcarbodiimide (45.13 mmol, 1.5 eq.), 1.4 g 4-dimethylaminopyridine (11.28 mmol, 0.38 eq.) and 100 mL dichloromethane. After stirring for 12 hours, a white precipitate formed, which was removed by filtration, and the filtrate was evaporated to yield a crude product as an off white powder. The crude product was dissolved and passed through a short plug of silica using hexane/ethyl acetate as eluent and finally recrystallized from hexane/ethyl acetate to yield 9.1 g of the octocrylene equivalent monomer.

### Synthesis of Octocrylene Equivalent Polymer

As indicated in FIG. 3, a Schlenk tube was charged with 1 g of monomer and 5 mL dichloromethane under an argon atmosphere. With vigorous stirring, a solution containing 2 mL of dichloromethane and 20 mg of Schrock's catalyst was rapidly injected into the Schlenk tube. The reaction mixture was vigorously stirred overnight then precipitated into 250 mL of vigorously stirred methanol. The solid polymer was collected via filtration, dried under vacuum, then dissolved in toluene and passed through a short plug of silica to remove catalyst residues. The collected solution was concentrated to a volume of approximately 10 mL and precipitated into 100 mL of vigorously stirred methanol. The solid polymer was collected by filtration and dried under vacuum to yield 810 mg of off white fibrous material.

### Synthesis of 1-(4-(tert-butyl)phenyl)-3-(4-hydroxyphenyl)propane-1,3-dione

A mixture of 6 g hexanethiol (50.94 mmol, 1.7 eq.) and 50 mL anhydrous dimethyl formamide was cooled to 5° C. in a round bottom flask under nitrogen atmosphere. 5.4 g potassium t-butoxide (47.94 mmol, 1.6 eq.) was added in one lot and the reaction mixture was allowed to warm to room temperature. After stirring for 20 min, 9.3 g Avobenzone (29.96 mmol) was added in one lot and the solution was heated to 110° C. for 1 h. The mixture was cooled to room temperature, poured into 100 mL of ice water, and finally acidified to pH 1 with 1 M aq. HCl. The resultant mixture was transferred to a separatory funnel and extracted twice using 75 mL portions of ethyl acetate. The organic layer was evaporated to yield 1-(4-(tert-butyl)phenyl)-3-(4-hydroxyphenyl)propane-1,3-dione as a pale brown oil (8.0 g), which was used without further purification.

### Synthesis of Avobenzone Equivalent Monomer

As indicated in FIG. 1, under a nitrogen atmosphere, a round bottom flask was charged with 8 g of 1-(4-(tert-butyl)phenyl)-3-(4-hydroxyphenyl)propane-1,3-dione, 4.6 g of 5-norbornene-2-carboxylic acid (mixture of isomers, predominantly endo, 33.74 mmol), 7 g dicyclohexylcarbodiimide (33.74 mmol), 1.6 g 4-dimethylaminopyridine (12.82 mmol) and 100 mL dichloromethane. After stirring for 12 hours, a white precipitate formed, which was removed by filtration, and the filtrate was evaporated to yield a crude product as a brown oil. The crude product was purified via silica gel chromatography using hexane/ethyl acetate (9:1) to yield 7.1 g of the avobenzone equivalent monomer as a white powder.

### Synthesis of Avobenzone Equivalent Polymer

As indicated in FIG. 3, a Schlenk tube was charged with 1 g of monomer and 5 mL dichloromethane under an argon atmosphere. With vigorous stirring, a solution containing 2 mL of dichloromethane and 20 mg of Grubbs' second generation catalyst was rapidly injected into the Schlenk tube. The reaction mixture was vigorously stirred overnight and precipitated into 250 mL of vigorously stirred methanol. Solid polymer was collected via filtration, dried under vacuum, then dissolved in toluene and passed through a short plug of silica to remove catalyst residues. The collected solution was concentrated to a volume of approximately 10 mL and precipitated into 100 mL of vigorously stirred methanol. The solid polymer was collected by filtration and dried under vacuum to yield 760 mg of off white fibrous material.

All patents, patent applications, provisional applications, and publications referred to or cited herein are incorporated by reference in their entirety, including all figures and tables, to the extent they are not inconsistent with the explicit teachings of this specification.

It should be understood that the examples and embodiments described herein are for illustrative purposes only and that various modifications or changes in light thereof will be suggested to persons skilled in the art and are to be included within the spirit and purview of this application.

We claim:

1. A UVA and/or UVB absorbing chromophore-comprising polymer, comprising a multiplicity of repeating units, wherein at least one of the repeating units comprises at least one UVA and/or UVB absorbing chromophore, and wherein independently the repeating units are derived from metathesis of a strained cycloakene, cycloalkadiene, bicycloalkene, or bicycloalkadiene, wherein optionally one or more sp³ hybridized carbons of the strained cycloakene, cycloalkadiene, bicycloalkene, or bicycloalkadiene is replaced with a heteroatom, and wherein the UVA and/or UVB absorbing chromophore-comprising polymer is soluble in at least one solvent.

2. The polymer of claim 1, wherein the UVA and/or UVB absorbing chromophore comprises an equivalent conjugated group derived from Aminobenzoic acid, Avobenzone, Cinoxate, Dioxybenzone, Homosalate, Menthyl anthranilate, Octocrylene, Octyl methoxycinnamate, Octyl salicylate, Oxybenzone, Padimate O, Phenylbenzimidazole sulfonic acid, Sulisobenzone, Trolamine salicylate, Ecamsule, Phenylbenzimidazole sulfonic acid, 4-Methylbenzylidene camphor, Bisoctrizole, Bemotrizinol, Bisdisulizole disodium, Drometrizole trisiloxane, Benzophenone-9, Ethylhexyl triazone, Diethylamino hydroxybenzoyl hexyl benzoate, Iscotrizinol, or Isoamyl p-Methoxycinnamate.

3. The polymer of claim 1, wherein one or more repeating units are from unsubstituted or substituted norbornene, norbornadiene, 7-oxo-norobornadiene, norbornene-2,3-dicarboxylic acid, norbornene-2,3 -dicarboxylic anhydride, or any norbornene-2,3 -dicarboxylic imide.

4. The polymer of claim 1, wherein the UVA and/or UVB absorbing chromophore-comprising polymer has a molecular weight distribution of less than 1.5.

5. The polymer of claim 1, wherein the UVA and/or UVB absorbing chromophore-comprising polymer is a random copolymer or block copolymer.

6. A UVA and/or UVB absorbing chromophore-comprising monomer, comprising a UVA and/or UVB absorbing chromophore and a strained cycloakene, cycloalkadiene, bicycloalkene, or bicycloalkadiene, wherein one or more sp³ hybridized carbons of the strained cycloakene, cycloalkadiene, bicycloalkene, or bicycloalkadiene is optionally replaced with a heteroatom.

7. The monomer of claim 6, wherein the UVA and/or UVB absorbing chromophore comprises an equivalent conjugated group derived from Aminobenzoic acid, Avobenzone, Cinoxate, Dioxybenzone, Homosalate, Menthyl anthranilate, Octocrylene, Octyl methoxycinnamate, Octyl salicylate, Oxybenzone, Padimate O, Phenylbenzimidazole sulfonic acid, Sulisobenzone, Trolamine salicylate, Ecamsule, Phenylbenzimidazole sulfonic acid, 4-Methylbenzylidene camphor, Bisoctrizole, Bemotrizinol, Bisdisulizole disodium, Drometrizole trisiloxane, Benzophenone-9, Ethylhexyl triazone, Diethylamino hydroxybenzoyl hexyl benzoate, Iscotrizinol, or Isoamyl p-Methoxycinnamate.

8. The monomer of claim 6, wherein the strained cycloakene, cycloalkadiene, bicycloalkene, or bicycloalkadiene is norbornene, norbornadiene, 7-oxo-norobornadiene, norbornene-2,3-dicarboxylic acid, norbornene-2,3-dicarboxylic anhydride, or any norbornene-2,3-dicarboxylic imide.

9. A method of preparing a UVA and/or UVB absorbing chromophore-comprising polymer comprising:
providing one or more UVA and/or UVB absorbing chromophore-comprising monomers of claim 6;
optionally providing one or more monomers comprising an unsubstituted or substituted strained cycloakene, cycloalkadiene, bicycloalkene, or bicycloalkadiene, wherein optionally one or more sp³ hybridized carbons of the strained cycloakene, cycloalkadiene, bicycloalkene, or bicycloalkadiene is replaced with a heteroatom;
providing a catalyst for ring-opening metathesis; and
optionally isolating the UVA and/or UVB absorbing chromophore-comprising polymer.

10. A method of preparing a UVA and/or UVB absorbing chromophore-comprising polymer comprising:
providing one or more monomers comprising an unsubstituted or substituted strained cycloakene, cycloalkadiene, bicycloalkene, or bicycloalkadiene, wherein optionally one or more sp³ hybridized carbons of the strained cycloakene, cycloalkadiene, bicycloalkene, or bicycloalkadiene is replaced with a heteroatom wherein a portion of the monomers have one or more first reactive functionalities, protected first reactive functionalities or a combination thereof;
providing a catalyst for ring-opening metathesis to polymerize the monomers;
optionally deprotecting the protected first reactive functionalities to yield first reactive functionalities;
combining one or more UVA and/or UVB absorbing chromophores comprising one or more second reactive functionalities that are complementary to the first reactive functionalities, wherein upon reaction between the first and second reactive functionalities a UVA and/or UVB absorbing chromophore-comprising polymer is formed; and
optionally isolating the UVA and/or UVB absorbing chromophore-comprising polymer.

11. A sunscreen lotion, comprising a UVA and/or UVB absorbing chromophore-comprising polymer of claim 1.

12. The sunscreen lotion of claim 11, further comprising a solvent.

13. A method of guarding against sunburn, comprising deposition of a UVA and/or UVB absorbing chromophore-comprising polymer of claim 1 on skin of a human subject.

14. The method of claim 13, wherein deposition comprises spreading the UVA and/or UVB absorbing chromophore-comprising polymer dispersed or dissolved in a fluid vehicle over the skin.
